(19) United States
(12) Patent Application Publication
(10) Pub. No.: US 2023/0331934 A1
(43) Pub. Date: Oct. 19, 2023

(54) NEW HYDROGELS

(71) Applicant: Leibniz-Institut für Neue Materialien gemeinnützige GmbH, Saarbrücken (DE)

(72) Inventors: Minye Jin, Saarbrücken (DE); Gülistan Kocer, Saarbrücken (DE); Julieta I. Paez, Saarbrücken (DE)

(21) Appl. No.: 18/028,782

(22) PCT Filed: Sep. 29, 2021
(86) PCT No.: PCT/EP2021/076842

§ 371 Date: Mar. 28, 2023

(30) Foreign Application Priority Data
Sep. 30, 2020 (DE) 10 2020 125 546.2

(51) Int. Cl. C08J 3/075; C12N 5/00; C08J 3/24; C08K 5/5313

(52) U.S. Cl. CPC C08J 3/075; C12N 5/0012; C08J 3/246; C08K 5/5313; C08J 2323/06

(57) ABSTRACT

Hydrogels are formed by the condensation of aromatic or heteroaromatic CN groups with aminothiol groups. Gelling takes place under physiological conditions, is biocompatible, and can be used for cell encapsulation.

### FIELD OF THE INVENTION

The invention relates to hydrogels, processes for production thereof and to use thereof.

### PRIOR ART

Hydrogels are three-dimensional networks of crosslinked hydrophilic polymers that comprise a high proportion of water. Such materials are known as matrix materials for biological applications such as active ingredient delivery, wound materials, tissue engineering and may also be used in cell culture. Due to their aqueous and porous structure, they allow nutrients to be transported easily to the cells.

Many natural or synthetic polymers have already been used to produce hydrogels, for example collagen, gelatin, polyethylene glycol (PEG). Different reactions and mechanisms have been investigated for crosslinking the hydrogels, for example photo-polymerization, Michael addition or similar.

The control of the cross-linking reaction in particular is a major challenge, especially when the hydrogel is to be produced to encase cells. If the gel polymerizes too rapidly, it is often not homogeneously crosslinked. If it polymerizes too slowly, the constituents to be enclosed, for example cells, may deposit and are not enclosed homogeneously.

### Problem

The object of the invention is to provide a process for producing a hydrogel which allows use in particular for encasing cells. It is also an object of the invention to provide a corresponding hydrogel and use thereof.

### Solution

This object is achieved by the inventions having the features of the independent claims. Advantageous developments of the inventions are characterized in the dependent claims. The wording of all claims is hereby made part of the content of this description by reference. The inventions also include all reasonable and in particular all recited combinations of independent and/or dependent claims.

A process for producing a hydrogel comprising the following steps:
a) producing a composition comprising
   a1) at least one macromer comprising at least two 1,2- or 1,3-aminothiol groups as functional groups,
   a2) at least one macromer comprising at least two aromatic or heteroaromatic groups as functional groups, each of which are substituted by at least one cyano group, wherein at least one component a1) or a2) comprises at least three of the functional groups mentioned;
   a3) at least one reducing agent without thiol groups;
b) reaction of the two macromers via the functional groups to form a hydrogel.

Individual process steps are described in detail hereinbelow. The steps need not necessarily be carried out in the stated sequence and the process to be described may also have further steps not mentioned.

A macromer is understood to mean a compound having an average molar mass of less than 500 kDa, preferably less than 100 kDa, in particular less than 50 kDa. The average molar mass is determined as the weight-average molecular weight using gel permeation chromatography (GPC).

Particular preference is given to macromers having an average molar mass of less than 50 kDa, in particular less than 30 kDa.

In a particular embodiment of the invention, the average molar mass of a macromer is between 100 Da and 500 kDa, preferably between 200 Da and 200 kDa, in particular between 800 Da and 100 kDa.

It is important here that the macromer bears the appropriate functional groups and that these are available for the reaction.

Preference is given here to macromers having 2, 3, 4, 5, 6, 7, 5 8, 9 or 10 functional groups, preferably 2, 3, 4, 5, 6, 7, 8 functional groups, particularly preferably 2, 3, 4, 5 or 6 functional groups, especially 2, 3 or 4 functional groups.

Hydrogel formation means that a hydrogel is formed by crosslinking. Sufficient crosslinking reactions therefore take place.

This can be controlled by the type and amount of the components used.

In a further preferred embodiment, at least one component a1) or a2) comprises at least 4 of the functional groups specified.

In a preferred embodiment, both components a1) and a2) comprise at least 3, preferably at least 4, of the functional groups specified. Both components a1) and a2) particularly preferably comprise 3, 4, 5, 6, 7, 8, 9 or 10 functional groups, preferably 3, 4, 5, 6, 7, 8 functional groups, particularly preferably 3, 4, 5 or 6 functional groups, especially 3 or 4 functional groups.

Preference is given to water-soluble macromers. This means that the macromers are in solution to the necessary extent under the conditions of the reaction.

Preference is given to macromers based on oligomers or polymers. They may be natural or synthetic oligomers or polymers. Examples of synthetic oligomers or polymers are poly(meth)acrylates such as poly(meth)acrylamides, poly(meth)acrylic acid, polyHPMA or polyHEMA, polyethylene glycol (PEG), polyvinyl alcohol (PVA), polyurethane (PU), polyvinylpyrrolidone (PVP), polyamides, poly(amidoamines) (PAMAM), polyesters, polylactides, polyglycolic acid (PGA) or poly(lactide-co-glycolide) (PLGA), polyanhydrides, poly(ortho)esters, polyacetals, poloxamers (block copolymers of ethylene oxide (PEG) and propylene oxide (PPG)) such as PEG-co-PPG-co-PEG), poly-2-oxazolines, polyphosphazenes, polyglycerol, polyamines such as polylysine or polyethyleneimine (PEI), polycarbonates, polyglutamic acid, especially poly-gammaglutamic acid, polyaspartic acid (PASA), polyphosphonates, or natural oligomers such as DNA, RNA, gelatine, polyhydroxyalkanoates (PHA), poly-gamma-glutamic acid, proteins or peptides such as collagens, VPM, albumin or fibrin, polysaccharides such as agarose, chitin, chitosan, chondroitin, mannan, inulin, dextran, cellulose, alginates or hyaluronic acid. Preference is given to oligomers based on polyethylene glycol. The oligomers and polymers are functionalized with the appropriate functional groups.

In the case of the peptide-based oligomers, the 1,2- or 1,3-aminothiol groups are preferably provided by the corresponding amino acids such as cysteine or homocysteine. Peptide-based means here that at least 80% of the molecular mass of the corresponding oligomer is composed of natural or non-natural amino acids. Such oligomers therefore comprise at least two aminothiol groups, in particular at least two cysteines. Preference is given here to terminal cysteines which are attached to the oligomer via the carboxyl group.

The at least partial use of natural polymers also allows the introduction of specifically cleavable sites in the hydrogel, for example by enzymes.

It may be necessary for the functional groups to be bonded to the oligomer or polymer via a short linker, for example via one or more esters, ethers or amide bonds. Preference is given to linkers having a molar mass of less than 5000 Da, preferably less than 1500 Da, preferably less than 800 Da, especially less than 500 Da or less than 200 Da.

The aminothiol groups are preferably present as free aminothiol groups. It is also possible that they are provided with groups which are cleaved off before formation of the hydrogel.

In the context of the invention, an aminothiol group is understood to mean a 1,2- or 1,3-aminothiol group which is preferably arranged on an aliphatic carbon skeleton. Examples of compounds comprising such groups are cysteine, homocysteine, penicillamine or 2-methylcysteine.

The macromers preferably have a molar degree of substitution of more than 80%, especially more than 90% (determined by 1H-NMR). This means that more than 80 or 90% of the suitable coupling points for functional groups have an appropriate functional group. A suitable coupling point is, in particular, a functional group at the end of a polymer chain, the degree of substitution being preferably based on all functional groups.

The macromers are preferably designed as arm-like polymers. This means that one or more branching point(s), for example one or more carbon atoms, are each the starting point for linear polymer chains, at the ends of which 1, 2, 3 or 4, in particular 1 or 2, especially one functional group is arranged. The respective polymeric chains are preferably not crosslinked with one another here. In the case of polyethylene glycols, the central branching point may be, for example, an ether based on tetrol, such as 1,2,3,4-butanetetrol or the tert-butyl derivative thereof, wherein the hydroxyl groups are etherified polyethylenes, at the end groups of which the functional groups are arranged, optionally via a linker.

The reducing agent is a reducing agent without thiol groups. This means that it has no thiol groups or precursors thereof. Reducing agents which are able to reduce dithiols under these conditions are preferred. These can be reducing carboxylic acids, sugars, uronic acids, aldehydes, formic acid or ascorbic acid, or phosphine-based reducing agents, for example THP (tris(3-hydroxypropyl)phosphine) or TCEP (tris(2-carboxyethyl)phosphine), preferably TCEP (tris(2-carboxyethyl)phosphine).

In a preferred embodiment, TCEP is used in the molar ratio of 0.5 to 2 equivalents of TCEP per aminothiol group, preferably 0.8 to 2 equivalents, preferably 0.9 to 1.5, in particular 1 to 1.2, especially preferably 1 equivalent.

The use of a reducing agent without thiol groups results in better crosslinking and in a reduced gelation time, especially under physiological conditions. Thus, the reduction is more chemoselective and avoids reactions with the thiol groups of the macromers. In combination with a high degree of substitution of the macromers, the gelation time can be significantly reduced and thus lead to a mechanically more stable gel at the same time.

The macromer a2) is a macromer comprising at least two aromatic groups which are each substituted with at least one cyano group. Preference is given to groups of the formula (1):

M-Ar—CN (1)

where Ar is an electron-deficient aryl group or electron-deficient heteroaryl group which may be substituted by one or more radicals R¹. This makes it possible to select reaction conditions under which the aminothiol groups of the first macromer can carry out a condensation reaction with the CN group to form a five- or six-membered ring.

M is a preferably covalent bond to the macromer and is preferably a single bond, ether, or carbonyl group. The carbonyl group can be part of an ester, carbamate, carbonate, or amide bond. Thus, the corresponding esters or amides can be used as the Ar group for coupling to the macromer, such as appropriately substituted benzoic acid esters or benzoic acid amides.

An aryl group in the context of this invention comprises 6 to 40 carbon atoms; a heteroaryl group in the context of this invention comprises 1 to 40 carbon atoms and at least one heteroatom, with the proviso that the sum of carbon atoms and heteroatoms is at least 5. The heteroatoms are preferably selected from N, O and/or S. An aryl group or heteroaryl group is understood to mean here either a simple aromatic ring, i.e. benzene or a simple heteroaromatic ring, for example pyridine, pyrimidine, thiophene etc. or a fused aryl or heteroaryl group, for example naphthalene, naphthalimide, benzothiazole, anthracene, quinoline, isoquinoline, benzothiazole etc.

An electron-deficient aryl group or heteroaryl group is understood to mean an aryl group or heteroaryl group the n-electron density of which is reduced by negative induction effects or negative mesomeric effects (-I effects or -M effects). A listing of substituents or groups that cause these effects can be found in any standard organic chemistry textbook. Examples of -I substituents include, without restriction: OH, halogens, especially fluorine and chlorine, NO₂, unsaturated groups; for -M substituents: NO₂, CN, aryl groups or heteroaryl groups. These electron-withdrawing groups (EWG) must of course be conjugated to the leaving group CN, i.e. in the ortho or para position in the case of carbocycles, in order to be able to exert the desired effect. In the case of heteroaryl groups, the heteroatoms contribute accordingly to the reduction of the electron density depending on their position. Two or more different groups may also be present.

Examples of electron-deficient aryl groups are nitrobenzenes, benzaldehydes, benzonitriles, benzoic acid esters, which may be further substituted by one or more groups R¹ as defined below. An example of such an aryl group are compounds based on nitrobenzoic acid having 1 or 2 nitro groups, for example nitrobenzoic acid esters or nitrobenzoic acid amides having a CN group at least in one position. This group is preferably disposed in the meta position to a nitro group. Particular preference is given to a nitro group in the 3-position and the CN group in the 4-position.

Examples of electron-deficient heteroaryl groups are, for example, mononuclear heteroaromatics such as pyridines, pyrimidines, pyrazines, pyridazines, triazines such as 1,3,5-triazine, 1,2,4-triazine or 1,2,3-triazine, tetrazines such as 1,2,4,5-tetrazine, 1,2,3,4-tetrazine or 1,2,3,5-tetrazine, oxazoles, iso-oxazole, thiazoles such as 1,2-thiazole or 1,3-thiazole, isothiazole, oxadiazoles such as 1,2,3-oxadiazole, 1,2,4-oxadiazole, 1,2,5-oxadiazole and 1,3,4-oxadiazole, thiadiazoles such as 1,2,3-thiadiazole, 1,2,4-thiadiazole, 1,2,5-thiadiazole or 1,3,4-thiadiazole, imidazole, pyrazole, triazoles such as in particular 1,2,4-triazole or 1,2,3-triazole, tetrazole, polynclear heteroaromatics such as quinolines, isoquinolines, naphthalimide, benzimidazole, benzoxazole, benzothiazole, benzopyridazine, benzopyrimidine, quinoxaline, benzotriazole, purine, pteridine, indolizine and benzothiadiazole, which may be further substituted by one or more groups Rl¹ as defined below.

Preferred heteroaryl groups are pyrimidine, quinoline and benzothiazole, especially benzothiazole, where the CN group is preferably located in the 2-position there.

In a preferred embodiment of the invention, Ar is a polynuclear heteroaryl group or a mononuclear heteroaryl group substituted with at least one further aryl group or heteroaryl group, preferably phenyl.

In a further embodiment, Ar is an aryl group bearing with at least one -I or -M substituent, preferably 1 or 2, preferably F or NO₂, particularly preferably NO₂.

R¹ is the same or different at each occurrence H, D, F, Cl, Br, I, N (R²)₂, CN, NO₂, OR², SR², C(═O)N(R²)₃, C(═O)N(R²)₂, C(═O)R², a straight-chain alkyl group having 1 to 20 carbon atoms or an alkenyl or alkynyl group having 2 to 20 carbon atoms or a branched or cyclic alkyl group having 3 to 20 carbon atoms, wherein in each case the alkyl, alkenyl or alkynyl group may be substituted by one or more radicals R₂, wherein one or more non-adjacent CH₂ groups may be replaced by R²C═CR², C≡C, C═O, NR², O, S, C(═O)O or C(═O)NR², or an aryl group or heteroaryl group which may in each case be substituted by one or more radicals R².

Preferably, R² is then the same or different, at each occurrence, H, D, F, OH, or an aliphatic, aromatic and/or heteroaromatic organic radical, in particular a straight-chain alkyl group having 1 to 20 carbon atoms, in which one or more H atoms may also be replaced by F.

In a preferred embodiment, R¹ is the same or different, at each occurrence, H, D, F, Cl, Br, I, N(R²)₂, CN, NO², OR², SR², C(═O)OR², C(═O)N(R²)₂, C(═O)R², a straight-chain alkyl group having 1 to 10 carbon atoms or an alkenyl or alkynyl group having 2 to 10 carbon atoms or a branched or cyclic alkyl group having 3 to 10 carbon atoms, wherein in each case the alkyl, alkenyl or alkynyl group may be substituted by one or more radicals R², wherein one or more non-adjacent CH² groups may be replaced by R²C═CR², C≡C, C═O, NR², O, S, C(═O)O or C(═O)NR², or an aryl group or heteroaryl group which may in each case be substituted by one or more radicals R².

Preferably, R² is then the same or different, at each occurrence, H, D, F, OH, or a straight-chain alkyl group having 1 to 5 carbon atoms, in which one or more H atoms may also be replaced by F or OH.

In a further preferred embodiment, R¹ is the same or different, at each occurrence, H, D, F, OH, C(═O)OH, a straight-chain alkyl group having 1 to 5 carbon atoms or an aryl group or heteroaryl group having 5 to 10 aromatic ring atoms, in which one or more H atoms bonded to carbon may also be replaced by F or NO₂.

In a preferred embodiment of the invention, at least one macromer is based on poly(meth)acrylates such as poly(meth)acrylamides, poly(meth)acrylic acid, polyHPMA or poly-HEMA, polyethylene glycol (PEG), polyvinyl alcohol (PVA), polyurethane (PU), polyvinylpyrrolidone (PVP), polyamides, poly(amidoamines) (PAMAM), polyesters, such as polylactides, polyglycolic acid (PGA) or poly(lactide-co-glycolide) (PLGA), polyanhydrides, poly(ortho)esters, polyacetals, poloxamers (block copolymers of ethylene oxide (PEG) and propylene oxide (PPG)) such as PEG-co-PPG-co-PEG), poly-2-oxazolines, polyphosphazenes, polyglycerol, polyamines such as polylysine or polyethyleneimine (PEI), polycarbonates, polyglutamic acid, especially poly-gamma-glutamic acid, polyaspartic acid (PASA), polyphosphonates, and the other macromer is based on DNA, RNA, gelatine, polyhydroxyalkanoates (PHA), poly-gamma-glutamic acid, poly-gamma-glutamic acid, peptides such as collagens, VPM, albumin or fibrin, polysaccharides such as agarose, chitin, chitosan, chondroitin, mannan, inulin, dextran, cellulose, alginates or hyaluronic acid. This makes it possible to integrate biochemical reactivity into the hydrogel, for example cleavage or degradability, for example by means of ester groups or carbonate groups in the macromer or by enzymatic reactions. Examples of suitable peptides are, for example, enzymatically cleavable dicysteine pep-tides such as VPM (sequence: CGRDVPMSMRGGDRK(C)G). These bear a free cysteine and thus a 1,2-aminothiol at the N-terminus and at the C-terminus respectively. The cysteine can also be disposed, in particular at the C-terminus, also on a side chain of another amino acid so that it can react as a 1,2-aminothiol, for example by bonding to the side chain of lysine. Such proteins can there-fore serve as linear crosslinkers if they have exactly two 1,2-aminothiol functions.

Both macromers are preferably used such that the number of aminothiol:CN functional groups of the two macromers that contribute to the crosslinking is from 2:1 to 1:2, preferably 1.5:1 to 1:1.5, particularly preferably 1.2:1 to 1:1.2, especially at 1:1. If two or more different compounds with the respective functional group are used, the figures refer to the total number of these groups, for example when using different compounds with aminothiol groups. For instance, one aminothiol compound may be used, for example, for modification and another compound for crosslinking.

Both macromers are preferably present in solution, preferably in aqueous solution. It may be necessary to adjust the pH, preferably by using a buffer.

In a preferred embodiment, a first solution is provided with the first macromer comprising aminothiol groups and a second solution is provided with the second macromer comprising the aromatic CN group. These two solutions are then combined with each other.

In a preferred embodiment, the pH of the macromer solutions used, in particular of the composition, is from 6 to 9 (at 25° C.). The pH is preferably adjusted by a buffer, preferably using a buffer concentration of between 5 mM and 200 mM. Examples of buffers are PBS or HEPES (2-(4-(2-hydroxyethyl)-1-piperazinyl)ethanesulfonic acid). A higher buffer concentration can stabilize the pH in the gel when using high macromer concentrations. Preference is given to a pH of 6 to 9, preferably 6.5 to 8.5, particularly preferably 6.6 to 8. This makes it possible to set the gelation time between, for example, 16 seconds (pH 8) to 27 seconds (pH 6.6) (measured at 25° C. at a constant macromer concentration). Despite the short gelation time, the coupling reaction allows good mixing and the production of homogeneous hydrogels.

In a further preferred embodiment, the macromer content in the composition is 1 to 30% by weight, preferably 3 to 15% by weight, particularly preferably 3 to 10% by weight, based on all macromers used.

The temperature during formation of the hydrogel is preferably between 20° C. and 45° C., preferably between 20° C. and 40° C.

The hydrogel formation reaction described here is characterized by several advantages. In contrast to known crosslinking reactions, it is neither particularly rapid nor particularly slow under physiological conditions. This enables the encapsulation of cells or other substances such as peptides, enzymes, chemical compounds or the like during formation of the gel. The composition remains viscous even longer during the formation of the gel, so that it can be mixed for even longer with low shear forces. This enables a homogeneous distribution of the cells in the hydrogel without the need for further steps, such as turning the gel during curing.

The proposed reaction is also sufficiently rapid under physiological conditions. This enables the use in cell cultures, preferably in three-dimensional cell culture or even in situ. Also, gelation can be controlled via pH, which enables the use for formation of gels in situ, for example in 3D printing, or in an organism when an appropriate composition is injected.

In addition, the presence of the reducing agent without thiol groups can avoid the formation of undesirable side reactions such as disulfides. Surprisingly, it was found that this side reaction can be suppressed very well especially when using TCEP. As a result, macromers having a very high content of reactive groups may also be used.

The reaction is also orthogonal to OH groups, amino groups, carboxylic acid groups and acrylate groups, which do not react under physiological conditions.

In a preferred embodiment of the invention, the reaction of the two macromers contributes exclusively to the formation of the hydrogel. No other crosslinking reactions take place.

The rate of reaction can be controlled by the choice of the aromatic or heteroaromatic group which bears the CN group, of the reducing agent and the pH. In this way, the rate of gelation can be adapted to the respective use.

The ratio of the two macromers is preferably selected such that all functional groups have reacted after the reaction. This may depend on whether further functionalizations are carried out.

For instance, it is possible, for example, to modify the second macromer by prior addition of aminothiol-containing compounds before crosslinking and formation of the hydrogel is initiated by adding the first macromer. As a result, the hydrogel may be modified with additional functions. For example, fluorophores or bioactive reagents are possible.

Examples of bioactive reagents are tissue growth promoters, chemotherapeutic agents, proteins (glycoproteins, collagen, lipoproteins), cell binding mediators, for example fibronectin, laminin, collagen, fibrin, or integrin-binding sequences (for example RGD or cadherin-binding sequences), growth factors, differentiation factors or fragments of the aforementioned reagents. Examples are epidermal growth factor EGF, endothelial growth factor VEGF, fibroblast growth factors such as bFGF, insulin-like growth factors (e.g. IGF-I, IGF-II), transforming growth factors (e.g. TGF-α, TGF-β), DNA fragments, RNA fragments, aptamers or peptidomimetics, preference being given to cell binding mediators such as VEGF.

The modification can be used, for example, to create appropriate environments in the hydrogel depending on the cells to be cultured.

The reagents are preferably used at effective concentrations, which may be, for example, in the range from 0.01 to 100 mM, preferably 0.1 mM to 50 mM, in particular 0.2 mM to 10 mM, especially 0.5 to 5 mM, based on the swollen gel.

The invention also relates to a composition for producing a hydrogel comprising at least two macromers a1) and a2) as described for the process.

The invention also relates to a hydrogel obtained with the process according to the invention.

The invention also relates to a hydrogel comprising a first plurality of macromers crosslinked to a second plurality of macromers, wherein the crosslinking is effected via a plurality of N,S-containing five- or six-membered rings attached to an Ar group, especially via 4,5-dihydrothiazoles attached to an Ar group at the 2-position, where Ar is an aromatic or heteroaromatic group.

Such a bond can be obtained from the reaction of a CN group with a 1,2- or 1,3-aminothiol group as described above. Advantageous embodiments are described for the process.

The hydrogels according to the invention are stable for a long time, preferably up to 6 weeks. They can be modified and obtained in a simple manner and under physiological conditions.

They are particularly suitable for encapsulating cells, for three-dimensional cell cultures, organoids, biomaterials, injectable biomaterials, cell therapies, tissue modification, tissue regeneration, tissue transplantation, regenerative medicine, 3D printing, 3D bioprinting, wound dressings or wound treatment, means of active ingredient delivery, in vitro models for studying or testing diagnostics or therapeutics or cell transplantations.

Due to the reaction under physiological conditions, the reaction specified can be used in particular in the biological field. For instance, it is conceivable, for example, that the two macromers reacting with each other and the reducing agent are only combined or mixed with each other in situ. This can be achieved, for example, by means of a multi-component syringe.

The invention relates to a process of encasing cells, wherein the hydrogel is formed in the presence of the cells in order to encase the cells. This can be used, for example, for cell culture, in particular for three-dimensional cell culture.

The invention also relates to a kit for producing a hydrogel comprising the macromers a1) and a2) as described for the process.

The reaction described is also suitable for additionally crosslinking existing gels. In such a process, a gel comprising at least two of the functional groups of component a1) or a2) is provided and reacted with a macromer having corresponding functional groups according to macromer a1) or a2), wherein in this case the macromers a1) or a2) have at least two of the functional groups so that crosslinking of the gel occurs by means of this reaction.

The invention therefore also relates to a process for modifying gels, comprising the steps of:
a) providing a gel or a precursor thereof, comprising at least two functional groups according to component a1) or at least two functional groups according to component a2);
b) adding a composition comprising at least one macromer in accordance with the respective other component, wherein the macromer has at least two functional groups;
c) modifying the gel or the precursor thereof by reaction of the functional groups.

The process is preferably used for subsequent modification after the gel has been produced. This makes it possible to modify the gel under physiological conditions, for example to adjust the mechanical parameters thereof.

Further details and features are apparent from the following description of preferred exemplary embodiments in conjunction with the subsidiary claims. The respective features may be realized here alone or in a plurality in conjunction with one another. The options for achieving the object are not limited to the exemplary embodiments. Thus for example, indicated ranges always comprise all—unlisted—intermediate values and all conceivable subintervals.

The exemplary embodiments are shown schematically in the figures. Identical reference numbers in the individual figures indicate here identical or functionally identical elements or elements that correspond to each other in terms of their functions. Details shown are:

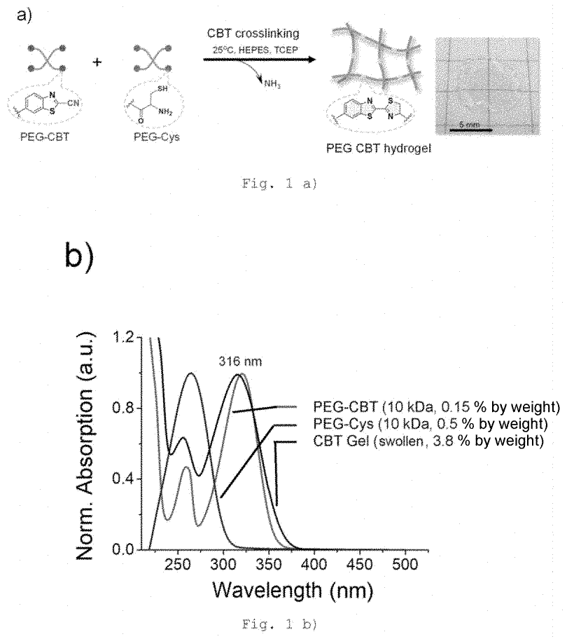

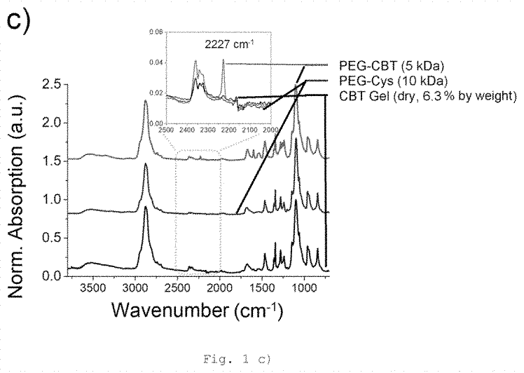

FIG. 1a) Formation of firefly-inspired PEG hydrogels by CBT ligation. a) Schematic representation of CBT crosslinking to form firefly-inspired hydrogels and image of a swollen CBT hydrogel.

FIG. 1b, 1c) b) UV/Vis characterization and c) FR-IR characterization of hydrogels formed vs. precursors.

FIG. 1d) Representative time-sweep curve showing shear storage (G′) and loss moduli (G″) as a function of time during in situ gelation.

FIG. 1e) Final G′ after swelling (black squares) and swelling ratio of CBT hydrogels at increasing polymer concentration (mean±SD shown, n=4, white circles). Conditions for FIG. 1b): PEG-CBT (0.15% by weight) and PEG-Cys (0.5% by weight) solutions and derived swollen CBT gel (3.8% by weight) in 20 mM HEPES pH 8. Conditions for FIG. 1c): undiluted solid macromers and derived dry CBT gel (6.3% by weight). Conditions for FIG. 1d) and e): 4A-20 kDa PEG, specified final polymer concentration in each case, in 20 mM HEPES buffer pH 8, t=2 h curing at T=25° C.;

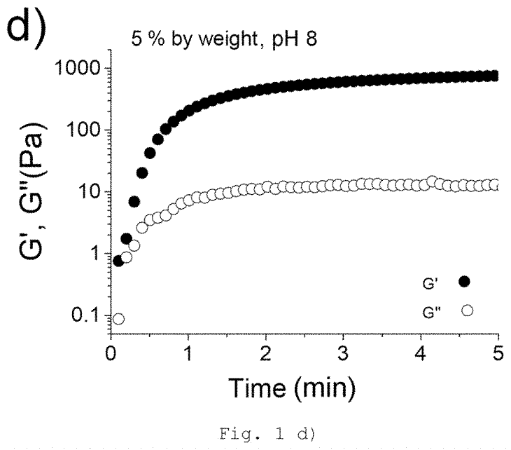

FIG. 2 Synthesis of PEG-CBT and PEG-Cys macromers. Reagents and conditions: i) K₂CO₃, dry DMF, 75° C., overnight; ii) thioanisole/trifluoroacetic acid (TFA), dichloromethane (DCM), room temperature, 1 h; iii), N-methylmorpholine (NMM), dry dimethylformamide (DMF), room temp., 3 d; iv) HBTU, HOBT, DIPEA, dry DMF, room temp., 3 d; v) TFA: triisopropylsilane (TIS): water (95: 2.5:2.5), room temp., 1.5 h;

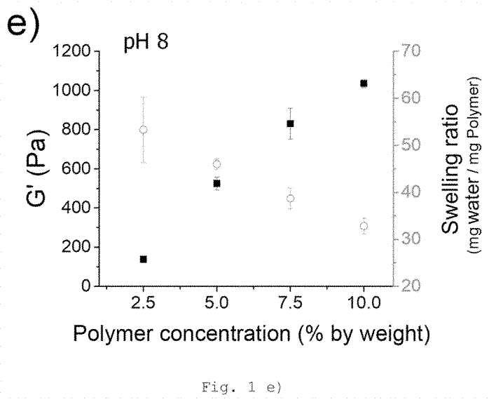

FIG. 3 pH modulation of gelation rate in CBT hydrogels. a) Shear modulus of in situ cured hydrogels. b) Shear modulus after swelling. Conditions: 4A, 20 kDa PEGS, 5% by weight polymer content, in 20 mM HEPES at 25° C. (mean±SD shown, n=4);

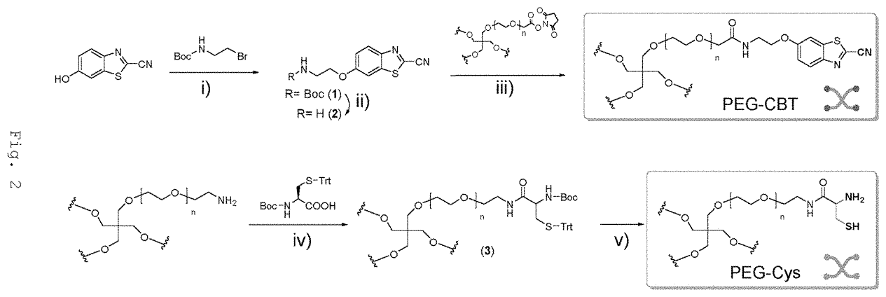

FIG. 4 Microscale homogeneity of CBT gels. a) Fluorescence confocal microscopy image of a fluorescently labeled CBT gel, scale bar=1 mm. b) Pixel intensity distribution of the CBT gel as a function of pixel spacing, corresponding to the cross section stated under a). Conditions: 4A, 20 kDa, 5% by weight gel, labeled with 0.01 mM Alexa-Fluor 350.;

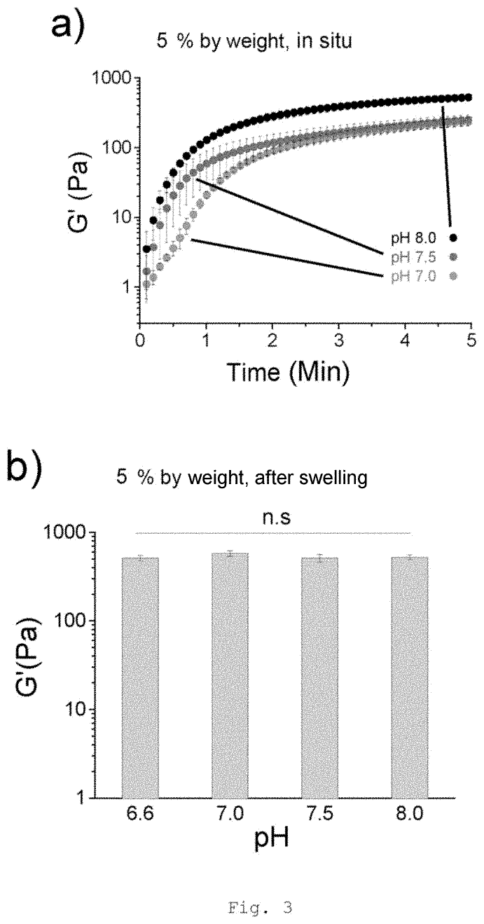

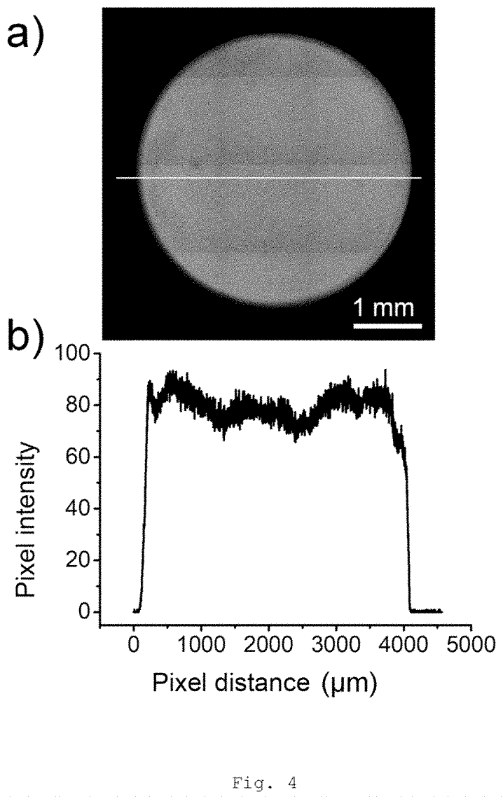

FIG. 5 Investigation of the hydrolytic stability of CBT gels using a gravimetric method. Conditions: 4A, 10 kDa, 5% by weight gels, incubated in RPMI cell culture medium (comprising 10% FBS and 1% P/S, pH 7.4) at 37° C. for 5 weeks (mean±SD shown, n=3);

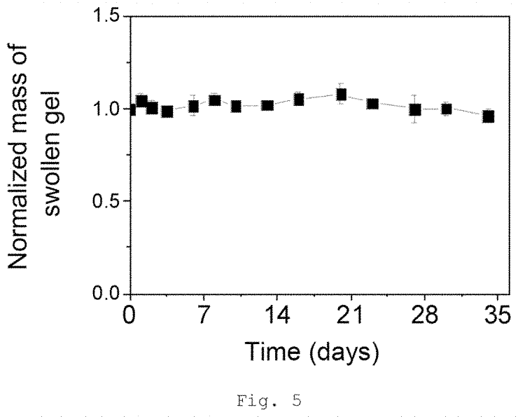

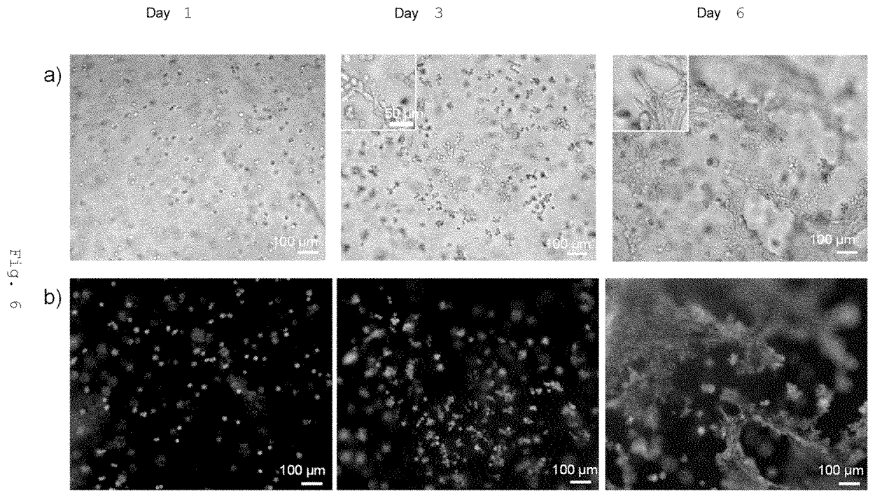

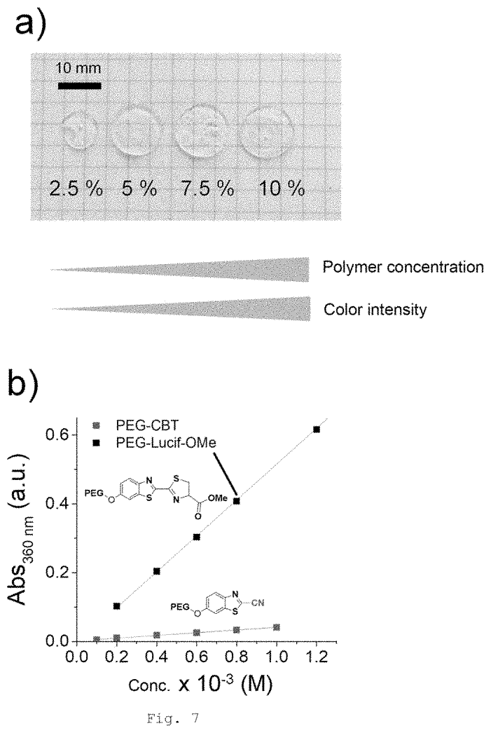

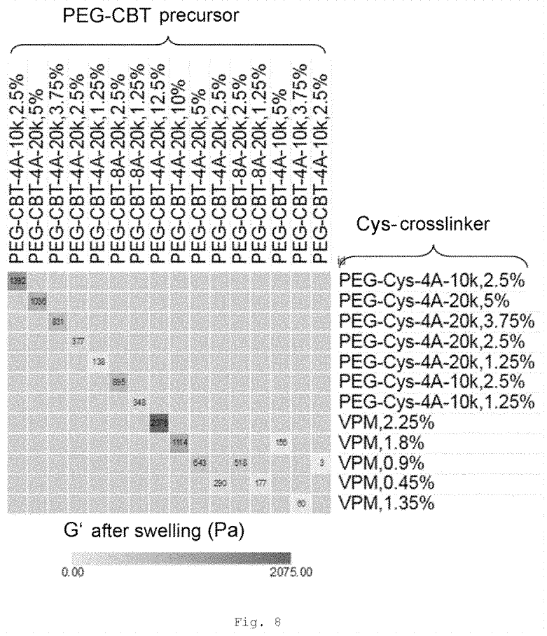

FIG. 6 Encapsulation of L929 fibroblasts in CBT hydrogels, functionalized with cell-adhesive cyclo(RGDfK(C)) peptide and crosslinked with cell-degradable VPM peptide, and culture for 1, 3 and 6 days. a) Bright-field micrographs and b) live (green)/dead (red) staining of these cells at the indicated culture time points, showing their viability after encapsulation. Scale bars: 100 μm. Final gel composition: 4A, 20 kDa, 4% by weight PEGCBT, 1 mM cyclo(RGDfK(C)), 3.14 mM VPM peptide; the cells were cultured in complete culture medium;

FIG. 7 Optical properties of CBT hydrogels. a) Photograph of 4A, 20 kDa CBT gels, with increasing polymer content. The gels show increased color intensity with increasing polymer concentration. b) Determination of the molar absorption coefficient (s) of the PEG-CBT macromer compared to the model PEG-luciferin-OMe macromer. The values are listed in Table 3.

FIG. 8 Adjustability of the mechanical strength of CBT gels within physiologically relevant values for 3D cell encapsulation. The heat map shows G′ after swelling for CBT gels with variable polymer content (1.25 to 12.5% by weight), precursor molar mass (10 vs. 20 kDa), multivalence (4A vs. 8A), and topology (4A vs. linear Cys crosslinker). Conditions: The gels were produced in the composition indicated with a constant CBT:Cys molar ratio (1:1) in 20 mM HEPES buffer pH 8.0, hardened at 25° C. for 2 h and swollen to equilibrium (24 h);

FIG. 9 Adjustability of the gelation time of CBT gels. The heat map shows the gelation time of CBT gels with variable polymer content (1.25 to 12.5% by weight), precursor molar mass (10 vs. 20 kDa), multivalence (4A vs. 8A), and topology (4A vs. linear Cys crosslinker). Conditions: The gels were produced in the composition indicated with a constant CBT:Cys molar ratio (1:1) in 20 mM HEPES buffer pH 8.0 at 25° C.;

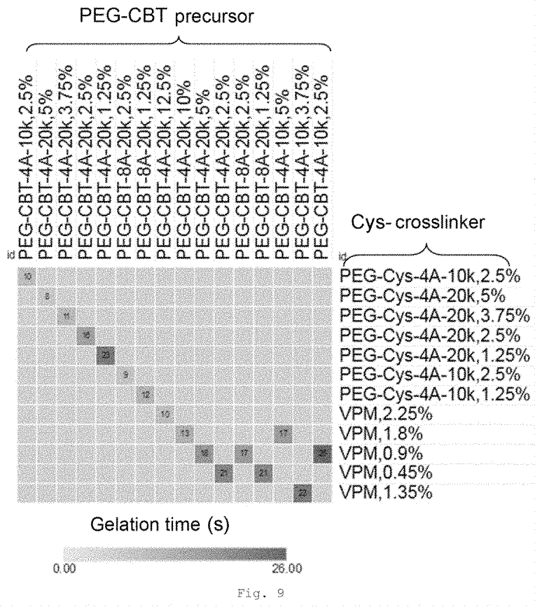

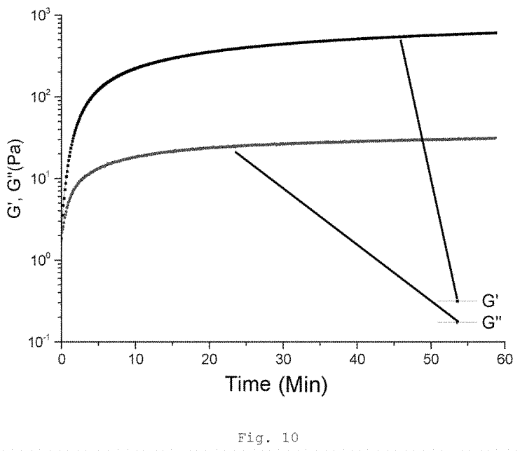

FIG. 10 Time-sweep curve showing shear storage (G′) and loss moduli (G″) as a function of time during in situ gelation (5% by weight 4A-10k-PEG-CBT and 10% by weight 4A20k-PEG-Cys; 20 mM HEPES+0.6% TCEP, pH=8.0; with ≥90% degree of substitution);

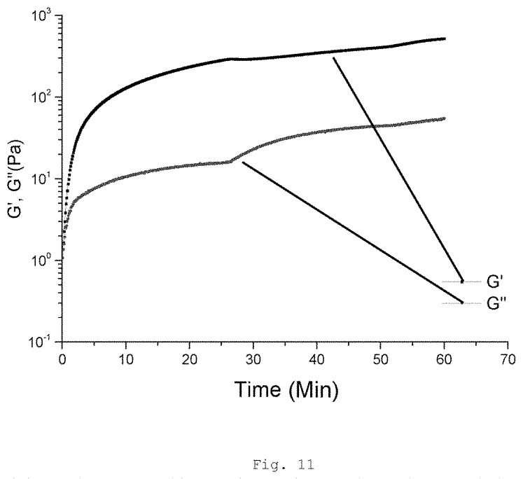

FIG. 11 Time-sweep curve showing shear storage (G′) and loss moduli (G″) as a function of time during in situ gelation (5% by weight 4A-10k-PEG-CBT and 10% by weight 4A-20k-PEG-Cys; 20 mM HEPES+0.6% TCEP, pH=8.0 with 65% degree of substitution);

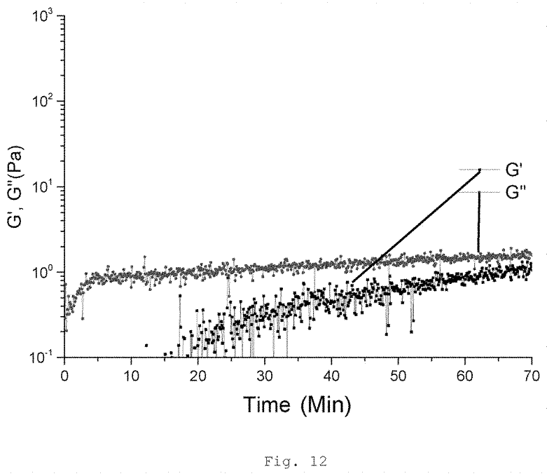

FIG. 12 Time-sweep curve showing shear storage (G′) and loss moduli (G″) as a function of time during in situ gelation (5% by weight 4A-10k-PEG-CBT and 10% by weight 4A20k-PEG-Cys; 20 mM HEPES+0.6% TCEP, pH=8.0 with ≤50% degree of substitution);

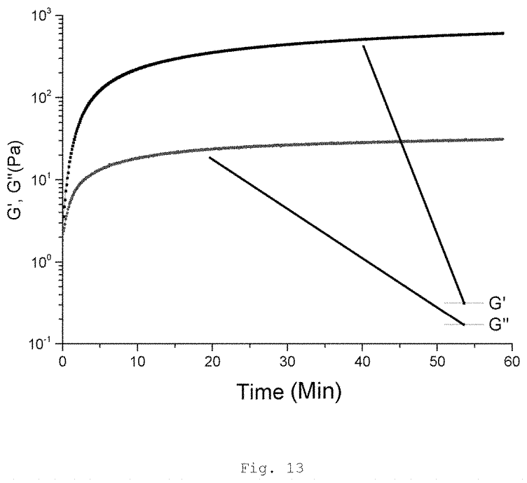

FIG. 13 Time-sweep curve showing shear storage (G′) and loss moduli (G″) as a function of time during in situ gelation (5% by weight 4A-10k-PEG-CBT and 10% by weight 4A20k-PEG-Cys; 20 mM HEPES+0.6% TCEP, pH=8.0; with ≥90% degree of substitution (DS));

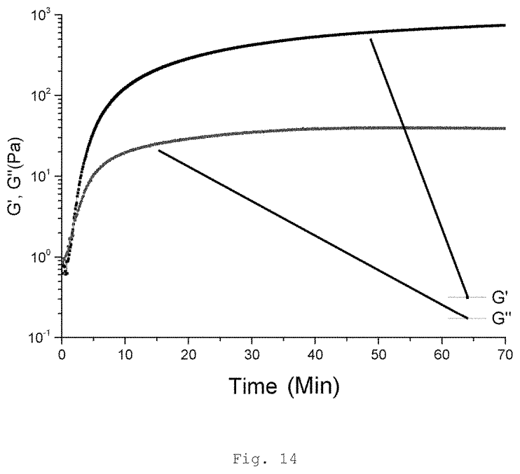

FIG. 14 Time-sweep curve showing shear storage (G′) and loss moduli (G″) as a function of time during in situ gelation (5% by weight 4A-10k-PEG-CBT and 10% by weight 4A20k-PEG-Cys; 20 mM HEPES+0.3% DTT, pH=8.0; with ≥90% degree of substitution);

### MATERIAL AND METHODS

Chemicals and solvents were used in p.a. quality. 4-arm (4A) and 8-arm (8A) (molecular masses 5, 10 and 20 kDa) star polyethylene glycol (PEG) polymers end-funtionialized with succinimidyl carboxylmethyl ester (PEG-NHS) groups or amino groups were obtained from Jenkem (USA). 2-Cyano-6-hydroxybenzothiazole was obtained from Fluorochem (UK). Linear VPM CGRDVPMSMRGGDRK(C)G and cyclo(RGDfK(C)) peptide sequences were purchased from GeneCust (FR). PEG-CBT and PEG-Cys macromers were prepared according to protocols described.

Buffer solutions of pH 8.0, 7.5, 7.0 and 6.6 were freshly prepared in each case. Buffer precursors were prepared as follows: CBT precursor was dissolved in 20 mM HEPES and Cys precursor was dissolved in 20 mM HEPES which contained 1 equivalent of tris(2-carboxyethyl)phosphine (TCEP) per Cys equiv. and 180 mM NaHCO3. A TCEP:Cys molar ratio of (1:1) was maintained to prevent disulfide formation between the free Cys groups. After dissolving the polymers in the appropriate buffer, the solutions were vortex mixed, treated with ultrasound (approx. 5 s) and centrifuged to eliminate bubbles. The final pH of the precursor solutions was verified with a pH meter (pH-1 micro, Presens, DE). The spectroscopic characterization of PEG-CBT and PEG-Cys precursors and derived CBT gels was carried out using NMR, FT-IR and UV/Vis.

### Estimation of the Gelation Time of CBT Hydrogels by a Macroscopic Test

A macroscopic test was carried out to estimate the gelation time of hydrogels in accordance with Paez, J. I.; Farrukh, A.; Valbuena-Mendoza, R.; W [symbol] odarczyk-Biegun, M. K.; del Campo, A. Thiol-Methylsulfone-Based Hydrogels for 3D Cell Encapsulation. ACS Applied Materials & Interfaces 2020, 12 (7), 8062-8072, DOI: 10.1021/acsami.0c00709. Precursor solutions at a specific concentration and at a specific pH were prepared as described above. 30 μL of the CBT precursor solution were placed in a plastic Eppendorf vial, after which 30 μL of the Cys precursor solution were added and a stopwatch was started. The curing solution was continuously mixed with a pipette (pipette tip size=2-200 μL, 53 mm; from Eppendorf epT.I.P.S.®, Germany) until the gelation solution stopped flowing. Time was measured using a Rotilabo-Signal-Timer TR 118 stopwatch (Roth, Germany). Gelation time was taken to be the time elapsed between mixing the two components and the time point at which pipetting of the mixture was no longer possible.

### Rheology of CBT Hydrogels in the Case of in Situ Crosslinking

The rheological properties of hydrogels were measured with a Discovery HR-3 Rheometer (TA Instruments, USA) using 12 mm thick parallel plates and a Peltier temperature control system, typically at 25° C. Precursor solutions were prepared as above. 20 μL of the CBT precursor solution were loaded onto the lower Peltier plate of the rheometer, followed by the addition of 20 μL of the Cys precursor solution and mixing with the pipette tip directly on the plate. The upper plate was brought nearer to achieve a gap size of 300 μm and the sample was sealed with paraffin or silicone oil to avoid evaporation during measurement unless otherwise stated. The total time for loading the sample and starting the measurement was about 60 s. Strain runs (0.1 to 1000% strain at a frequency =1 Hz) and frequency runs (0.01 to 100 Hz at a strain =1%) were carried out to determine the linear viscoelastic regime. Time run measurements were carried out within the linear viscoelastic regime using the following parameters: initial gap of 300 μm, controlled axial force (0.0±0.1 N), frequency 1 Hz, strain 1%, temperature=25° C., unless otherwise stated. To capture the first moments of the gelation processes of fast-curing CBT gels, additional time-sweep measurements were carried out without using an oil trap; as a result, the time required for stopping the experiment could be reduced to 30 s. Such time sweep measurements were only carried out for 5 min to avoid drying effects. The gelation time was estimated from the time sweep curves as the time point when G′=G″.

### Rheology of CBT Hydrogels After Swelling

25 μL of the CBT precursor solution were placed in a cylindrical PDMS mold (6 mm diameter), mixed rapidly with 25 μL of the Cys precursor solution and crosslinked in a humid chamber at room temperature for 2 h. The resulting gels were carefully demolded and swollen for 24 h in 20 mM HEPES at the appropriate pH. The swollen hydrogels (ca. 8 mm diameter) were loaded into the rheometer and measured using an 8 mm diameter top plate geometry having a rough surface to ensure good contact with the swollen gel. Time sweep measurements were carried out for 3 min to avoid evaporation of the sample using the following parameters: controlled initial axial force 0.05 N, variable initial gap (depending on the thickness of the sample, typically 700-1000 μm), frequency 1 Hz, strain 1%, temperature=25° C.

### Statistical Analysis

The data were expressed as mean±standard deviation (SD). For each condition, 3 to 4 independent experiments were carried out. One-way analysis of variance (ANOVA) with a Tukey test of variance was used to determine the statistical significance between the groups. Statistical analysis was carried out to compare different groups and the significant difference was set at *p<0.05.

Swelling Ratio of CBT Hydrogels

Following the above procedure, 4A, 20 kDa, 10, 7.5, 5, 2.5% by weight CBT gels at pH 8 were prepared in a PDMS mold and cured for 2 h at room temperature. The resulting hydrogels were carefully demolded and swollen in 20 mM HEPES at room temperature for 24 h and the mass of the swollen gel was measured (Mₛ). The gel was freeze-dried and the mass of the dry hydrogel was measured (M_{d}). The swelling ratio (SR) was calculated according to equation S1 and expressed in mg water per mg polymer:

$$S R = \frac{{M}_{s} - {M}_{d}}{{M}_{d}} \qquad ( Equation S1 )$$

The experiments were carried out in triplicate. The data were expressed as mean±SD.

### Hydrolytic Stability of CBT Hydrogels

Following the above procedure, 4A, 10 kDa, 5% by weight CBT gels at pH 8 with a total volume of 50 μL were prepared in a mold. The resulting hydrogels were equilibrated in Milli-Q water (24 h, 37° C.) and then in medium (RPMI cell culture medium pH 7.4 containing 10% fetal bovine serum (FBS) and 1% penicillin/streptomycin (P/S) (24 h, 37° C.)). The initial mass of the swollen hydrogel was measured (Mᵢ). Then, the hydrogel was placed in a 24-well plate and incubated in RPMI cell culture medium (3 mL) at 37° C. for 5 weeks. At selected time points, the gel was removed from the medium, the excess liquid was gently blotted from the hydrogel surface using a KimWipe®, and the sample mass was measured (Mₜ). Fresh medium was refilled after each measurement. The normalized mass of the swollen gel when incubated in the medium was followed over time and calculated according to Equation S2:

Normalized mass of swollen gel=Mₜ/Mᵢ (equation S2)

The experiments were carried out in triplicate. The data were expressed as mean±SD.

### Microscale Homogeneity of CBT Hydrogels

4A-20 kDa, 5% by weight PEG-Cys solution (43.2 μL) was fluorescently labeled by coupling 0.5 mM Alexa Fluor 350 maleimide

(AF350, Life Technologies) (1.8 μL) at 37° C. for 15 min. Then, 4A, 20 kDa 5% by weight PEG-CBT (5 μL) was spotted in a plastic μ-Slide Angiogenesis (Ibidi, DE), followed by addition of the labeled PEG-Cys solution (5 μL) to the same well and mixing of the precursors. The curing mixture was cured at 37° C. for 15 minutes and HEPES buffer was added to the well. The final gel composition consisted of 5% by weight polymer, 0.01 mM fluorophore. The gels were imaged using a Zeiss LSM 880 confocal microscope with a 10× air objective. The image was recorded in tile mode (5×5 images with 10% overlap) in the center of the gel with respect to the z-direction. Image analysis was carried out with Image J (NIH). To investigate the homogeneity of the shaped gels, profile plots of dye-labeled gels were drawn in Image J (plot profile command) with a one-dimensional region of interest (line) for at least three different gel samples, with the pixel intensities given along the gel diameter (distance). For visualization and readability purposes, the brightness of the images has been adjusted where necessary. Raw images (gray values of intensity) were used for data processing.

### Cell Studies

### Cell Culture

The fibroblast cell line L929 (ATCC) was cultured at 37° C. and 5% CO₂ in complete medium (RPMI 1640 (Gibco, 61870-010), supplemented with 10% FBS (Gibco, 10270), 100 U mL⁻¹ penicillin and 100 μg mL⁻¹ streptomycin (Invitrogen), in accordance with Takeuchi, A.; Hayashi, H.; Naito, Y.; Baba, T.; Tamatani, T.; Onozaki, K. Human Myelomonocytic Cell Line THP-1 Produces a Novel Growth-promoting Factor with a Wide Target Cell Spectrum. Cancer Research 1993, 53 (8), 1871-1876. For the encapsulation experiments, L929 cells were counted and resuspended in serumfree medium to reach a final cell density of 20 000 cells per gel.

### PEG Hydrogel Preparation For 3D Cell Culture

Precursor solutions of 4A, 20 kDa PEG-CBT (100 mg mL⁻¹, 10% by weight) were prepared by dissolving the lyophilized polymer in sterile 20 mM HEPES buffer pH 8.0 in a sterile laminar flow and used directly without further filtration. Solutions of cyclo(RGDfK(C)) (3.5 mg mL⁻¹, 5 mM) and VPM peptide (31.9 mg mL⁻¹, 17.5 mM) were prepared in sterile HEPES buffer pH 8.0 with 1 equiv. of TCEP per Cys equiv. and 178 mM NaHCO₃. These concentrations were kept constant during all cell experiments. PEG-CBT stock solution (4 μL, 10% by weight) was mixed with cyclo(RGDfK(C)) (2 μL, 5 mM) and incubated for 30 min at 37° C. The fibroblast cell suspension (1×10⁶ cells mL⁻¹, cell density within the typical range of 3×10⁵-3×10⁷ cells mL⁻¹) in serum-free RPMI medium (2 μL) was added to the above solution, and 8 μL of the resulting mixture was placed in an Ibidi 15-μwell angiogenesis slide. Immediately, the VPM peptide solution (2 μL, 17.5 mM) was added to the p-well, mixed thoroughly with the pipette tip and allowed to crosslink. The final gel composition consisted of 4% by weight PEG-CBT, 1 mM Cyclo(RGDfK(C)), 3.5 mM VPM, 8 mM TCEP and 72 mM NaHCO₃. The CBT hydrogels were allowed to polymerize for 15 minutes at 37° C. and 5% CO₂. After gelation, complete RPMI medium (45 μL) was added to remove residual TCEP for 10 min, the medium was replenished and the culture maintained for up to 6 days, exchanging the medium every other day.

### Live/Dead Test

All experiments were carried out in triplicate. L929 fibroblasts were cultured in CBT hydrogels for 1-6 days and the cell culture medium was removed. The samples were incubated with fluorescein diacetate (40 μg mL⁻¹) and propidium iodide (30 μg mL⁻¹) in PBS for 5 minutes, washed twice with PBS and imaged with the Zeiss Axio Observer microscope with appropriate filter settings and using a 10× air objective. The cells were stored in PBS and imaged under normal cell culture conditions (at 37° C. and 5% CO₂ in a humidified environment) in a climate chamber connected to the microscope within 1 h after staining. The excitation parameters were adjusted to use minimal light intensity in order to maintain cytocompatibility. For each sample, imaging was carried out over different z-stacks in three different wells per condition, and at least 300 single cells were manually counted using ImageJ to calculate the percentage viability of each sample.

### Experimental Results

CBT hydrogels were developed from 4A star PEG precursors having a molar mass of 10-20 kDa. PEG-CBT macromers were synthesized in three steps (FIG. 2) from commercial 2-cyano-6-hydroxybenzothiazole by the Williamson ether reaction, followed by acid cleavage of the Boc group and coupling of the amine group of the intermediate to a commercial PEG-NHS ester. PEG-CBT macromers having a degree of substitution >93% (measured by end group determination method by means of 1H-NMR) were obtained on a 0.35 g scale in overall moderate to excellent yields (46-99%). The PEG-Cys macromer was synthesized in two steps (FIG. 2) by coupling Boc-Cys(Trt)-OH-amino acid to PEG-amine, followed by acid cleavage of the protecting groups. PEG-Cys was obtained on a 0.5 g scale in high yields (90-99%) and had a degree of substitution of >90%. The PEG-CBT precursor was very stable during storage as evidenced by ¹H-NMR. No decomposition was observed on the solid compound stored in a refrigerator for at least 6 months, and the aqueous solutions of the macromer remained unchanged over at least 1 month storage at room temperature. The good stability of the precursors is a relevant aspect of hydrogels according to the invention.

The CBT ligation-mediated formulation of CBT hydrogels was carried out under conditions typically used for the preparation of cell-encapsulating hydrogels. 4A, 20 kDa precursor solutions were prepared at a concentration of 5% by weight in 20 mM HEPES buffer pH 8.0. Both precursor solutions were mixed in a (1:1) molar ratio of CBT:Cys at 25° C. and hydrogel formation was observed within 16 s as estimated by a macroscopic test (Table 1). This is an advantageous time that allowed the two precursor solutions to mix well and resulted in hydrogels that appeared transparent and homogeneous to the naked eye (a representative image of a swollen gel is shown in FIG. 1a)). In addition to 20 mM HEPES buffer, the crosslinking agent contained 1 equivalent of TCEP per Cys groups and 90 mM NaHCO₃. TCEP is a known reducing agent commonly used in biology laboratories to cleave disulfide bonds in the presence of living cells. Although previous reports of CBT ligation for bioconjugation applications have typically used (2:1) TCEP:Cys molar ratio, we sought to lower the TCEP concentration as much as possible to reduce the potential cytotoxicity of our formulation. We found that a molar ratio of (1:1) TCEP:Cys is sufficient to prevent disulfide formation, so this ratio was used for subsequent studies. In addition, the incorporation of TCEP into our formulation caused a pH decrease in the buffer solutions; however, this effect could be easily compensated by the addition of 90 mM NaHCO₃ to the working buffer. Sodium bicarbonate is a biocompatible base that is commonly used as a constituent of cell culture media.

Interestingly, the PEG-Cys and PEG-CBT solutions were colorless and pale yellow, respectively, while the derived CBT gels were pale yellow and this color intensified with increasing curing time and with increasing polymer content (see FIG. 7a)). To investigate this observation, the formation of CBT gels was evaluated spectroscopically. A thin CBT gel film was prepared between two quartz glass slides, rinsed with buffer and the UV/Vis spectrum thereof recorded (FIG. 1b)). The UV/Vis spectrum of the CBT gel showed an absorption band at λ_{Max}=316 nm, while the PEG-Cys precursor showed λ_{Max}=264 nm and the PEG-CBT precursor showed λ_{Max}=320 nm. Although λ_{Max} of the CBT gel was not redshifted compared to the PEG-CBT precursor, the former showed increased absorbance at λ>340 nm, presumably due to the formation of luciferin-like adducts as crosslinking points. To confirm this observation, a model macromer containing luciferinlike adducts was synthesized, the UV/Vis profile thereof recorded in the same buffer, and the molar absorption coefficient determined and compared to the PEG-CBT precursor. For this purpose, PEG-CBT and PEG-luciferin-OMe macromere were dissolved in 20 mM HEPES buffer (pH 8.0) at room temperature to achieve functional group concentrations of 0.1 to 1 mM and 0.17 to 1.4 mM respectively. The solutions were transferred to a quartz cuvette (optical path b=0.1 cm) and the UV absorbance was recorded in a wavelength range between 200-500 nm. The absorbance (Abs) as a function of concentration (c) was plotted and fitted to a linear function according to the Lambert-Beer law (Abs=ε*b*c). The molar absorption coefficient (ε) was calculated from the slope of the curve.

The molar absorption coefficient of PEG-luciferin at 360 nm was found to be 13-fold higher than that of PEG-CBT (see FIG. 7 and Table 3). A higher molar absorption coefficient at higher wavelengths of luciferin-like derivatives would explain the color change observed for CBT gels relative to the PEG-CBT precursor and the increase in gel color intensity as a function of polymer content.

To further characterize the formation of gels by CBT ligation, FT-IR experiments were carried out. FT-IR spectroscopy recorded over a rinsed and dried CBT gel showed the absence of the stretching vibration of the —CN group at 2227 cm⁻¹, which was originally present in the PEG-CBT precursor (FIG. 1c)). This confirms the consumption of the —CN group during the crosslinking reaction. Overall, these results spectroscopically support the formation of CBT gels through luciferin-like crosslinks.

In order to investigate the gelation kinetics and the final mechanical strength of CBT gels, oscillatory rheology was applied downstream of the gelation process. 5% by weight solutions of 4A-20 kDa PEG-CBT and PEG-Cys precursors in 20 mM HEPES buffer pH 8.0 were mixed directly on the rheometer at the same volume and in the molar ratio CBT:Cys (1:1) and the evolution of shear storage modulus (G′) and loss modulus (G″) was monitored over time at 25° C. Typical curves were obtained which corresponded to a fast curing mixture: Already at the beginning of the experiment, a gel formed which signified G′>G″ (FIG. 1d)). This indicates that the gelation time for this formulation is <30 s (note that the estimated time for mixing the solutions and setting up the experiment was ca. 30 s), in agreement with the values estimated by the macroscopic test from Table 1. As the curing process continued, G′ increased with time, reaching ˜760 Pa within 5 min. These results demonstrate the efficient curing of CBT gels under mild conditions.

The mechanical strength of the gel was also measured after swelling. In a separate experiment, hydrogels of the above formulation were prepared in a PDMS mold, allowed to cure for 2 h at 25° C. in a humid chamber to prevent evaporation, swollen to equilibrium (24 h) in the same buffer and the final mechanical strength was measured rheologically. A swollen hydrogel having a polymer content of 5% by weight showed a shear modulus G′=526 Pa (FIG. 1e), which is lower than G′ of the in situ gel (i.e. before swelling). Note that the measured swelling ratio for this composition was 46 mg of water per mg of polymer (FIG. 1e). By increasing the polymer concentration in the composition from 2.5 to 10% by weight, G′ increased linearly from 140 to 1040 Pa after swelling, while the swelling ratio decreased linearly from 53 to 33 mg of water per mg of polymer (FIG. 1e). These trends would indicate a linear increase in the crosslink density with the polymer concentration, consistent with a gelation process occurring through a step-growth mechanism involving two complementary functional groups. In addition to varying the polymer content, further adjustment of the mechanical strength of the gel (keeping the gelation medium constant, 20 mM HEPES buffer pH 8) was achieved by reducing the molar mass of the macromer from 20 to 10 kDa, by increasing the multivalency of the macromer (from 4A to 8A) and by combining a 4A macromer with a linear crosslinker of 1.8 kDa. Under these test conditions, the G′ measured after swelling ranged from 60 Pa to 2080 Pa (see heat map plot in FIG. 8), which corresponds to a modulus of elasticity E=180 to 6240 Pa (taking into account a Poisson's ratio of ca. 0.5 for PEG hydrogels). This range of swollen gel elasticity is consistent with the reported values for natural soft tissue and synthetic hydrogels used for successful 3D cell culture applications. These results show that CBT gels having physiologically relevant mechanics can be conveniently produced. The gelation time in all these formulations was <1 minute in this case (see FIG. 9).

In general, the rate of CBT ligation decreases with decreasing pH in the interval from 8 to 6 due to the lower thiolate-thiol ratio (pKa-thiol group ˜8). This trend is typical for thiol-mediated coupling reactions, which proceed via polar mechanisms and can be exploited for pH regulation of the gelation rate of CBT gels. Preliminary macroscopic tests carried out at a polymer concentration of 5% by weight showed that the gelation time increases from 12 s to 27 s when the pH decreases from 8 to 6.6 (Table 2). Time-sweep experiments conducted in situ confirmed the slower gelation rate when pH decreased from 8 to 7, which is characterized by the slower development of G′ with curing time (FIG. 3a). To confirm whether pH also influences the final mechanical strength of the material, CBT gels were prepared at different pH, cured for 2 h and G′ measured after swelling. The G′ values ranged from 514 to 580 Pa and no significant differences were found between the different pH groups (FIG. 3b). These results demonstrate the possibility of pH modulating the gelation kinetics of CBT hydrogels within the physiological values without altering their ultimate mechanical strength.

Although the hydrogel structure may appear homogeneous at the macro level (for example to the naked eye), gels having a gelation rate that is too rapid (i.e. a few seconds) are known to have an inhomogeneous microstructure due to insufficient mixing of the precursors. Such hydrogels typically exhibit microscopically small areas of high and low crosslink density, and previous work has shown that this inhomogeneity affects the reproducibility of the reaction of encapsulated cells. To examine the microscale homogeneity of CBT gels, fluorescently labeled hydrogels were prepared at a polymer concentration of 5% by weight on a culture plate, allowed to cure as described above, and imaged using confocal microscopy to determine the distribution of fluorescence intensity across the hydrogel (FIG. 4). The confocal visualization of the labeled CBT gel showed a uniform fluorescence space (FIG. 4a), indicating that the crosslinking density of the gel appears homogeneous at the microscale. This effect was confirmed by following the intensity distribution over the cross section of the gel and finding a variation of <20% (FIG. 4b). These results indicate that the gelation time of such a formulation=16 s allows good mixing of the precursors and this results in the production of homogeneous microscale CBT hydrogels. It is expected that the homogeneity of the material results in a more reproducible response of the encapsulated cells. The hydrolytic stability of CBT gels was evaluated using a grayimetric method under incubation conditions relevant to biomedical applications. 5% by weight CBT gels prepared in a mold were subjected to incubation in cell culture medium containing serum proteins at 37° C. and the mass of the swollen gel monitored over time. FIG. 5 shows that the normalized mass of the swollen gel remained virtually unchanged under the conditions tested: 96% of the mass was retained after 5 weeks of incubation. This result demonstrates the high hydrolytic stability of the CBT gels, similar to the stability reported for thiol-vinylsulfone-based and thiol-methylsulfonyl-based hydrogels. In addition, this offers the possibility to introduce controlled degradation properties, for example by the specific incorporation of enzymatically cleavable sequences into one of the gel precursors.

The possibility of using CBT gels as cell-encapsulating matrices was tested. 20 kDa PEG-CBT macromer was biofunctionalized with the cell-adhesive peptide Cyclo(RGDfK(C)) and crosslinked in 20 mM HEPES pH 8.0 with the enzymatically degradable VPM peptide in the presence of L929 fibroblast cells. The molar ratio of CBT: Cys was maintained (1:1) and the final gel composition was 4% by weight PEG-CBT, 1 mM Cyclo(RGDfK(C)) and 3.14 mM VPM. Such a composition provides a good balance between the mechanical, adhesive and degradative properties of the gel to support the 3D cell culture of fibroblast cells. After 15 minutes of curing, a cell-loaded gel was obtained, which was rinsed twice with cell culture medium to remove unreacted compounds, by-products and used TCEP. After 1, 3 and 6 days of culture, the gel samples were stained for the live/dead test and cell viability was quantified.

Successful cell encapsulation, which is characterized by high cell viability, was observed at all time points (FIG. 6). After 1 day of culture, a cell viability of >90% was found. This indicates that CBT gels are cytocompatible and cell viability is not adversely affected by either the CBT ligation by-product (ammonia) or the TCEP used as reducing agent in the PEG-Cys precursor solution. Under the conditions tested, a maximum total concentration of 8 mM ammonia is expected as a by-product of the condensation reaction between CBT and Cys groups. It should be noted that ammonia is a naturally occurring metabolite in mammals that is produced via various biosynthetic pathways. Ammonia is also a by-product of ostensibly enzymatically crosslinked hydrogels used for cell culture and tissue engineering applications. Transglutaminases catalyze the formation of a covalent isopeptide bond between carboxamide and amine groups from glutamine and lysine side chains, with release of ammonia, without appreciable in vitro toxicity. In addition, 8 mM TCEP was used during the gelation time (15 min) to prevent disulfide formation between free Cys groups. The concentration and the contact time between the cells and the reducing agent in our experiments are close to the concentration range (5 mM) and time (15 min) previously used for the reduction of disulfide bonds on the surface of leukocyte cells. After CBT gelation, the gels were rinsed once with cell culture medium; and it is expected that this step eliminated the ammonia generated and the residual TCEP from the formulation. This would explain the good cytocompatibility observed in our cell studies.

After 3 d of culture, the cells not only remained highly viable (ca. 94%), but also recognized the cell-adhesive peptide, as evidenced by cell protrusions and spreading. At 6 d culture, the high cell viability was maintained, which was qualitatively observed as the cells colonized the gel to a high degree and also reformed extensive cell-cell contacts with protruding cells, which prevented individual cell counting. At this time point, when the cells were imaged throughout the gel, a small amount of dead cells compared to highly colonized live cells was associated with interior regions of growing cell clusters (FIG. 6b).

It is known that the introduction of a cell-degradable peptide into otherwise non-degradable gels is required for cell elongation and spreading through locally mediated hydrogel remodeling by cell-secreted enzymes. Although we have not quantified the extent of cell elongation and spread here, the observations of cell protrusion formation and spread on day 3 and day 6, and cell growth, can be attributed to cell-mediated remodeling (together with recognition of cell-adhesive peptides) in these CBT gels, facilitated by VPM-peptide crosslinkers. In fact, some gel degradation was observed on visual inspection, particularly on day 6, although this was not yet complete and an intact piece of gel was still visible. Overall, these results demonstrate that

CBT gels are convenient matrices for cell encapsulation of fibroblasts up to 6 days. They support the attachment and proliferation of cells.

The hydrogel crosslinking according to the invention is based on the chemoselective condensation reaction between cyanobenzothiazole and free cysteine groups and takes place under physiological conditions. CBT hydrogels are derived from precursors that are easy to prepare and stable on storage, a key aspect for wide application.

CBT ligation enabled the preparation of hydrogels with convenient gelation time (<1 min), homogeneous structure on a microscale, and with adjustable mechanics within physiologically relevant values. The gelation time can be modulated by the working pH close to the physiological range without altering the mechanical strength of the final gel, thanks to the versatility of this crosslinking reaction.

In addition, CBT gels are hydrolytically stable and cytocompatible. CBT hydrogels with cell adhesive, cell degradable and mechanical stability have been formulated and tested for 3D cell culture. CBT gels are convenient matrices for cell encapsulation applications as they support cell culture for 6 days.

### Effect of the Degree of Substitution of the Precursor on the Material Properties

The degree of substitution of the precursors has a major influence on the gelation time and the final mechanical strength of he hydrogel. This was demonstrated by the preparation of hydrogels from a PEG-CBT precursor having a variable degree of substitution (>90% (FIG. 10), 65% (FIGS. 11) and 40% (FIG. 12)). For instance, a high degree of substitution (>90%) leads to more efficient gelation (lower gelation time) and higher mechanical strength of the gel (higher G′ value) (Table 4).

### Effect of the Reducing Agent on the Material Properties: DTT vs. TCEP as Reducing Agent

The reducing agent has an influence on the gelation kinetics of the system. This was verified by preparing hydrogels with different reducing agents, either DTT (dithiothreitol, FIG. 14) or TCEP (tris(2-carboxyethyl)phosphine, FIG. 13). Table 5 shows that using TCEP results in more efficient gelation (lower gelation time) than using DTT. The mechanical strength of the gel is similar (same G′ value).

### General Methods

The reagents were purchased from Fluorochem (Derbyshire, UK), Fluka (Taufkirchen, DE), Merck (Darmstadt, DE), ABCR (Karlsruhe, DE), AcrosOrganics (Geel, BE), Sigma-Aldrich (Steinheim, DE) and Carbolution (St. Ingbert, DE). The solvents were of p.a. purity and were used as purchased unless otherwise stated. 4-arm (4A) and 8-arm (8A) polyethylene glycol (PEG) polymers, molar mass=5, 10 and 20 kDa; functionalized with amine (PEG-NH2) or succinimidyl carboxymethyl ester (PEG-NHS), were purchased from Jenkem (USA).

The buffer solutions were freshly prepared. 20 mM HEPES buffer (pH 8.0; 7.5, 7.0 and 6.6) with and without TCEP were used. Deuterated solvents were purchased from Deutero GmbH (Kastellaun, DE). Deuterated phosphate buffered saline (d-PBS) with pD=7.6 (pH=8.0) was prepared by dissolving the correct amount of disodium phosphate, monosodium phosphate, sodium chloride and potassium chloride in D₂O; then the pD was adjusted with 40% DCl solution (Merck) or 40% NaOD solution until the desired pD value was reached.

Thin-layer chromatography (TLC) plates (ALUGRAM® SIL G/UV254) and silica gel for column chromatography (60Å pore size, 63-200 μm particle size) were purchased from Macherey-Nagel (Duren, DE). TLC plates were observed under 254 or 365 nm light. HPLC analysis and purification of the compounds were carried out on a JASCO 4000 (JP) HPLC equipped with a diode array, a UV-Vis detector and a fraction collector. Reprosil C18 columns were used for semi-preparative (250×25 mm) and analytical (250×5 mm) runs. Solvent gradients were used with a combination of the following eluents: Solvent A (MilliQ water+0.1% TFA) and solvent B (95% ACN/5% MilliQ water+0.1% TFA), typically over a period of 40 minutes. Purification of the modified polymers was typically carried out by dialysis against acetone and water. Spectra/Por 3 dialysis tubes (molecular weight cut-off MWCO=3.5 kDa) from Spectrum Chemical (USA) were used.

The 1H-NMR and ¹³C-NMR spectra of the solution were recorded at 25° C. on a Bruker Avance 300 MHz or on a Bruker Avance III UltraShield 500 MHz. The latter was equipped with a He-cooled 5 mm TCI-CryoProbe, a proton-optimized triple resonance ‘inverse’ NMR probe with external water cooling (CP TCI 500S2, H-C/N-D-05 Z). Unless otherwise stated, all measurements were carried out at 298 K and the residual solvent peak (7.25 ppm for CDCl₃, 2.05 ppm for acetone-d6, 2.50 ppm for DMSO-d6 and 4.79 ppm for D₂O) was used as internal reference. Chemical shifts (5) are reported in parts per million. The following abbreviations are used: s-singlet, d-doublet, t-triplet, q-quartet, m-multiplet, dd-doublet of doublets. The degree of substitution of the PEG polymer was calculated by end group determination. The integral of the signal corresponding to the PEG backbone (3.70-3.40 ppm) was set at 110 H, 220 H or 440 H (for a 5, 10 and 20 kDa macromer respectively) and compared with the integral of the protons corresponding to the incorporated molecule. In all cases, degrees of functionalization of >90% and yields of >85% were achieved. The data were analyzed with MestReNova.

Electrospray ionization mass spectrometry (ESI-MS) was recorded using a 1260 Infinity Liquid Chromatography/Mass Selective Detector (LC/MSD) (Agilent Technologies, DE) and quadrupole time-of-flight (Q-TOF) using a 6545 Accurate-Mass Quadrupole Time-of-Flight (LC/Q-TOF-MS) (Agilent Technologies, DE) using electrospray ionization. Matrix-assisted laser desorption/ionization time-of-flight (MALDI-TOF) mass spectrometry was recorded with an AB Sciex 4800 (Sciex-Company, DE) in linear mode in the mass range of 4000-40 000 Da. For sample preparation, dithranol (1,8,9-anthracenetriol) was used as the matrix and acetonitrile, MilliQ water, and THF as solvent. Formic acid was added to improve ionization. About 4800 individual recordings were accumulated for a spectrum for each sample.

The molar mass of the PEG precursors was characterized by gel permeation chromatography (GPC). The GPC system consisted of a Waters 515 HPLC pump (Waters, Milford, U.S.A.), three GRAM PSS (Mainz, DE) columns in series (GRAM 30, GRAM 100, GRAM 100), a Waters 2410 refractive index detector, a Waters 2487 UV detector (operating λ=260 nm). A PEG standard kit with a molar mass of 7, 12, 26 and 44 kDa (Jenkem USA) was used for the calibration, and DMF with 1 g L⁻¹ LiBr was used as eluent. The runs were carried out at T=60° C., flow rate=1 mL min⁻¹, polymer concentration=2.1 mg mL⁻¹ in DMF.

The UV/VIS spectra were recorded with a Varian Cary 4000 UV/VIS spectrometer (Varian Inc. Palo Alto, U.S.A.). FT-IR spectroscopy was recorded with a Bruker Vertex 70 spectrometer in absorption mode with film-fused samples using a diamond-attenuated total reflection (ATR) accessory.

### Chemical Synthesis

### Synthesis of tert-butyl (2-((2-cyanobenzo[d]thiazol-6yl)oxy)ethyl)carbamate (1):

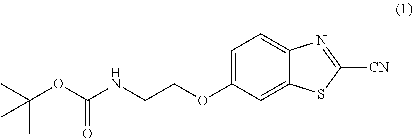 [SMILES: CC(C)(C)OC(=O)NCCOc1ccc2nc(C#N)sc2c1]

2-Cyano-6-hydroxybenzothiazole (0.78 g, 4.45 mmol, 1 equiv.) was dissolved in dry DMF (20 mL), followed by the addition of 2(Boc-amino)ethyl bromide (2 g, 8.9 mmol, 2 equiv.) and K₂CO₃ (1.23 g, 8.9 mmol, 2 equiv.) as solids. The mixture was stirred overnight at 75° C. The reaction course was monitored by analytical HPLC until the starting reagent had been completely consumed. The reaction was quenched by adding water (20 mL) and the aqueous layer was extracted four times with EtOAc. The combined organic layers were washed twice with saturated NaHCO₃ solution, water and brine, dried over MgSO4, filtered and evaporated. The crude product was purified by silica gel column chromatography (40% EtOAc in hexane) to obtain 0.66 g of the pure compound as a white solid. (Yield=46%). Analytical HPLC (Method: 30B-95B, 320 nm): elution time=28 min.

ESI-MS+: 320.0 (M+H). ¹H-NMR (300 MHz, acetone-d6, δ [ppm])=8.12 (1H, d, —CH Ar); 7.81 (1H, d, —CH Ar); 7.34 (1H, dd, —CH Ar); 6.28 (1H, m, —NH-amide); 4.22 (2H, t, —CH₂); 3.57 (2H, t, —CH₂); 1.40 (9H, s, -tBu). ¹³C-NMR (75 MHz, acetone-d6, δ [ppm])=160.65; 156.70; 147.78; 138.58; 134.47; 126.36; 119.83; 114.10; 105.39; 78.92; 68.63; 40.45; 28.59.

### Synthesis of 6-(2-aminoethoxy)benzo[d]thiazole-2-carbonitrile (2)

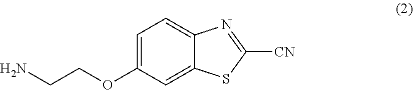 [SMILES: N#Cc1nc2ccc(OCCN)cc2s1]

Compound 1 (0.16 g, 0.50 mmol, 1 equiv.) was dissolved in dry DCM (8 mL) and cooled to 0° C. Thioanisole (1.1 mL, 10.0 mmol, 20 equiv.) was added and the mixture was stirred at 0° C. for 3 minutes. TFA (1.1 mL) was slowly added to the reaction vessel. The mixture was warmed to room temperature and stirring continued. The course of the reaction was monitored by TLC (40% EtOAc in hexane) until the starting reagent had been completely consumed (ca. 1 h). The crude oil was evaporated to reduce volume and added dropwise into cold diethyl ether. The precipitate obtained was isolated by centrifugation, purified by preparative HPLC (method: 5B-95B, 320 nm) and freeze-dried to obtain 74 mg of compound 2 as a white solid (yield=67%). Analytical HPLC (Method: 30B-95B, 320 nm): elution time=18 min. Q-ToF+: 220.1 (M+H). ¹H-NMR (300 MHz, acetone-d6, δ [ppm])=8.13 (1H, d, —CH Ar) ; 7.87 (1H, d, —CH Ar) ; 7.36 (1H, dd, —CH Ar); 4.61 (2H, t, —CH₂); 4.36 (2H, t, —CH₂). 13C-NMR (75 MHz, acetone-d6, δ [ppm])=159.92; 148.23; 138.60; 135.15; 126.56; 119.86; 114.13; 105.94; 66.57; 47.62.

### General Protocols for the Synthesis of PEG Macromers

A typical polymer modification procedure for a 4A, 10 kDa PEG macromer is described below. A similar process was followed for the preparation of macromers of different multivalency (8A) or different molar mass (5 or 20 kDa).

### Synthesis of PEG-CBT

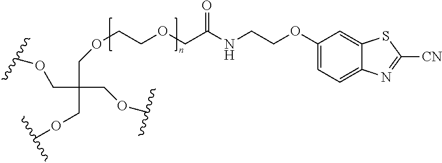

Compound 2 (294 μmol, 75 mg) and N-methylmorpholine (735 μmol, 81 μL) were dissolved in dry DMF (4 mL), purged with nitrogen and stirred for 15 min. 10kDa, 4-armed PEG-NHS (350 mg, 35 μmol) was dissolved in dry DMF (4 mL) and added to the above solutionunder a nitrogen flow. The mixture was stirred at room temperature under an inert atmosphere for three days, then dialyzed in acetone and water and freeze-dried. A white solid polymer was obtained and characterized by 1H-NMR in DCM-d2. The degree of functionalization was calculated to be 90%. Yield=85%. The polymer prepared in this way proved to be stable after >6 months of storage (evidenced by no changes in the ¹H-NMR spectrum). ¹H-NMR (500 MHz, DCM-d2, δ [ppm])=8.10 (d, —CH Ar); 7.46 (d, —CH Ar); 7.40 (m, —NH); 7.27 (dd, —CH Ar); 4.18 (t, —CH2); 3.97 (s, —CH₂C═O PEG); 3.83 (t, —CH₂); 3.80-3.35 (m, PEG core).

### Synthesis of PEG-Cys(Trt)-Boc

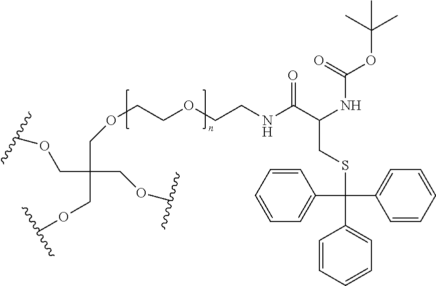

HBTU (82 mg, 1 equiv. based on COOH), HOBT (32 mg, 1 equiv. based on COOH), DIPEA (196 μL, 5.6 equiv. based on COOH) and Boc-Cys(Trt)-OH (59 mg, 201 μmol, 10 equiv.) were dissolved in dry DMF (2 mL) and added to a solution of PEG-amine (19.6 μmol, 196 mg) in dry DMF (2 mL). The mixture was stirred at room temperature for 2 days. The crude material was evaporated to reduce volume and added dropwise into cold diethyl ether. The precipitate obtained was isolated by centrifugation, dried under vacuum and characterized by ¹H-NMR in DCM-d2. The degree of functionalization was determined to be 98%. 1H-NMR (500 MHz, DCM-d2, δ [ppm])=7.95 (s, —NH); 7.43-7.23 (m, —CH Ar, Trt); 6.52 (s, NH); 4.94 (s, —CH chiral); 3.84-3.31 (m, PEG chain); 2.58-2.46 (m, —CH2) ; 1.40 (s, -tBu, Boc).

### Synthesis of PEG-Cys

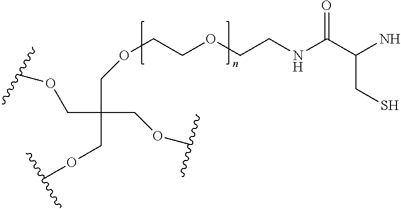

The protected macromer PEG-Cys(Trt)-Boc (197 mg) was dissolved in (95:2.5:2.5) TFA:TIS:water mixture (3 mL) and reacted at room temperature for 1.5 h. The crude oil was evaporated under nitrogen flow to reduce the volume and added dropwise into cold diethyl ether. The precipitate obtained was isolated by centrifugation, then dialyzed in acetone and water and freeze-dried. A white solid polymer was obtained, which was characterized by ¹H-NMR in DCM-d2, proving the complete removal of the protecting groups, and the degree of functionalization was calculated to be >99%. Yield=95%.

¹-NMR (500 MHz, DCM-d2, δ [ppm])=8.13 (s, —NH); 4.33-4.29 (t, —CH chiral); 3.74-3.45 (m, PEG chain); 3.42-3.39 (m, —CH₂).

TABLE 1
Gelation time of CBT hydrogels with increasing
polymer content measured by a macroscopic test.

Test.
Final polymer content (% by weight)

| | 2.5 | 5.0 | 7.5 | 10.0 |
|---|---|---|---|---|
| Gelation time^{a)} | 23 s | 16 s | 11 s | 8 s |

^{a)}The experiments were carried out on 4A-20 kDa macromers, in 20 mM HEPES buffer pH 8.0, T = 25° C. The gelation time was taken as the time that elapsed from the time point when the two components (30 μL each) were mixed until the mixture was pipetted. Pipette tip size = 2-200 μL, 53 mm.

TABLE 2
Gelation time of 5% by weight CBT hydrogels
at varying pH measured by a macroscopic test.

pH

| | 6.6 | 7.0 | 7.5 | 8.0 |
|---|---|---|---|---|
| Gelation time ^{a)} | 27 s | 24 s | 19 s | 16 s |

^{a)} The experiments were carried out on 4A-20 kDa macromers at 5% by weight polymer concentration, in 20 mM HEPES buffer pH 8.0, T = 25° C. The gelation time was taken as the time that elapsed from the time point when the two components (30 μL each) were mixed until the mixture was pipetted. Pipette tip size = 2-200 μL, 53 mm.

TABLE 3
Determination of the molar absorption coefficient
of PEG-CBT and model PEG-luciferin-OMe macromers.

| | Slope 360 nm = εxb | ε360 nm | Rel. |
|---|---|---|---|
| Macromer | [M⁻¹]^{a)} | [M⁻¹ cm⁻¹]^{b)} | value |
| PEG-CBT | 39.64 | 396 | 1.0 |
| PEG-Lucif- | 521.24 | 5210 | 13.2 |
| OMe | | | |

^{a)}determined from the plot of absorbance as a function of concentration, at λ = 360 nm, in 20 mM HEPES buffer pH 8, 25° C.
^{b)}calculated according to the Lambert-Beer law, taking into account the optical path b = 0.1 cm.

TABLE 4
Effect of the degree of substitution of the PEG-CBT precursor on the
gelation kinetics and the final mechanical strength of the hydrogel

| Degree of sub- | | | | |
|---|---|---|---|---|
| stitution of | | | | |
| the PEG-CBT | Gelation time | | G′ at t = 30 | G′ at t = 60 |
| precursor (a) | (min) (b) | | min (c) | min (c) |
| >90% | <1 | minute | 446 Pa | 610 Pa |
| 65% | 1.1 | min. | 294 Pa | 520 Pa |

| | | | |
|---|---|---|---|
| 40% | no gel | no gel | no gel |

(a) Gel composition: 4A-10 kDa-PEG-CBT (5% by weight, variable degree of substitution) + 4A-20 kDa-PEG-Cys (10% by weight, degree of substitution >90%) in 20 mM HEPES buffer pH 8.0, 1 equivalent of TCEP per Cys, 25° C.
(b) measured by in situ rheology as the crossing point between G′ and G″.
(c) G′ = shear storage modulus measured by in situ rheology. Conditions: Parallel plate 12 mm diameter, elongation 1%, frequency 1 Hz, 25° C.

TABLE 5
Influence of the reducing agent (TCEP vs. DTT) on the gelation
kinetics and the final mechanical strength of hydrogels.

| | | |
|---|---|---|
| Reducing | Gelation time | G′ at t = 30 |
| agent (a) | (min) (b) | min (c) |

| | | | |
|---|---|---|---|
| TCEP | <1 | minute | 446 Pa |
| DTT | 2.7 | min | 427 Pa |

(a) Gel composition: 4A-10 kDa-PEG-CBT (5% by weight, degree of substitution >90%) + 4A-20 kDa-PEG-Cys (10% by weight, degree of substitution >90%) in 20 mM HEPES buffer pH 8.0, 1 equivalent of TCEP or DTT per Cys, 25° C.
(b) measured by in situ rheology as the crossing point between G′ and G″.
(c) G′ = shear storage modulus measured by in situ rheology. Conditions: Parallel plate 12 mm diameter, elongation 1%, frequency 1 Hz, 25° C.

1. A process for producing a hydrogel comprising:
a) producing a composition comprising
a1) at least one macromer comprising at least two 1,2- or 1,3-aminothiol groups as functional groups,
a2) at least one macromer comprising at least two aromatic or heteroaromatic groups as functional groups, each of which are substituted by at least one cyano group, wherein at least one component a1) or a2) comprises at least three of the functional groups mentioned;
a3) at least one reducing agent without thiol groups; and
b) reaction of the two macromers via the functional groups to form a hydrogel.

2. The process as claimed in claim 1, wherein the macromer has an average molar mass of less than 500 kDa.

3. The process as claimed in claim 1, wherein the macromers have 2, 3, 4, 5, 6, 7, 8, 9 or 10 functional groups.

4. The process as claimed in claim 1, wherein the macromers are based on oligomers or polymers, for example poly(meth)acrylates such as poly(meth)acrylamides, poly(meth)acrylic acid, polyHPMA or polyHEMA, polyethylene glycol (PEG), polyvinyl alcohol (PVA), polyurethane (PU), polyvinylpyrrolidone (PVP), polyam ides, poly(amidoamines) (PAMAM), polyesters, polylactides, polyglycolic acid (PGA) or poly(lactide-co-glycolide) (PLGA), polyanhydrides, poly(ortho)esters, polyacetals, poloxamers (block copolymers of ethylene oxide (PEG) and propylene oxide (PPG)) such as PEG-co-PPG-co-PEG), poly-2-oxazolines, polyphosphazenes, polyglycerol, polyamines such as polylysine or polyethyleneimine (PEI), polycarbonates, polyglutamic acid, especially poly-gammaglutamic acid, polyaspartic acid (PASA), polyphosphonates, DNA, RNA, gelatine, polyhydroxyalkanoates (PHA), poly-gamma-glutamic acid, proteins or peptides such as collagens, VPM, albumin or fibrin, polysaccharides such as agarose, chitin, chitosan, chondroitin, mannan, inulin, dextran, cellulose, alginates or hyaluronic acid.

5. The process as claimed in claim 1, wherein the functional groups of the macromer a2) are functional groups of the formula (1):
M-Ar—CN (1)
wherein:
Ar is an electron-deficient aryl group or electron-deficient heteroaryl group which may be substituted by one or more radicals R¹;
M is the linkage to the macromer;
R¹ is the same or different at each occurrence H, D, F, Cl, Br, I, N(R²)₂, CN, NO₂, OR², SR², C(═O)OR², C(═O)N(R²)₂, C(═O)R²,a straight-chain alkyl group having 1 to 20 carbon atoms or an alkenyl or alkynyl group having 2 to 20 carbon atoms or a branched or cyclic alkyl group having 3 to 20 carbon atoms, wherein in each case the alkyl, alkenyl or alkynyl group may be substituted by one or more radicals R², wherein one or more non-adjacent CH₂ groups may be replaced by R²C═CR², C≡C, C═O, NR², O, S, C(═O)O or C(═O)NR², or an aryl group or heteroaryl group which may in each case be substituted by one or more radicals R²;
R² is the same or different at each occurrence H, D, F, OH, or an aliphatic, aromatic and/or heteroaromatic organic radical, in particular a straight-chain alkyl group having 1 to 20 carbon atoms, in which one or more H atoms may also be replaced by F.

6. The process as claimed in claim 5, wherein Ar is selected from the group consisting of nitrobenzenes, benzaldehydes, benzonitriles, benzoic acid esters, pyridines, pyrimidines, pyrazines, pyridazines, triazines, tetrazines, oxazoles, isooxazole, thiazoles, isothiazole, oxadiazoles, thiadiazoles such as 1,2,3-thiadiazole, 1,2,4-thiadiazole, 1,2,5-thiadiazole or 1,3,4-thiadiazole, imidazole, pyrazole, triazoles, tetrazole, quinolines, isoquinolines, benzimidazole, benzoxazole, benzothiazole, benzopyridazine, benzopyrimidine, quinoxaline, benzotriazole, naphthalimide, purine, pteridine, indolizine and benzothiadiazole, where Ar may in each case be substituted by one or more R¹ groups.

7. The process as claimed in claim 1, wherein the macromer content in the composition is 1 to 30% by weight.

8. The process as claimed in claim 1, wherein gelation takes place under physiological conditions.

9. A hydrogel obtained as claimed in claim 1.

10. A hydrogel comprising a first plurality of macromers crosslinked to a second plurality of macromers, wherein the crosslinking is effected via a plurality of N,S-containing five- or six-membered rings attached to an Ar group, where Ar is an aromatic or heteroaromatic group.

11. A composition for producing a hydrogel comprising the components a1) and a2) as claimed in claim 1.

12. A kit for producing a hydrogel comprising the components a1) and a2) as claimed in claim 1.

13. The use of a hydrogel as claimed in claim 9 for encapsulating cells, for three-dimensional cell cultures, organoids, biomaterials, injectable biomaterials, cell therapies, tissue modification, tissue regeneration, tissue transplantation, regenerative medicine, 3D printing, 3D bioprinting, wound dressings or wound treatment, means of active ingredient delivery, in vitro models for studying or testing diagnostics or therapeutics or cell transplantations.

14. A process for modifying gels, comprising:
a) providing a gel or a precursor thereof, comprising at least two functional groups according to component a1) or at least two functional groups according to component a2);
b) adding a composition comprising at least one macromer as claimed in claim 1 in accordance with the respective other component, wherein the macromer has at least two functional groups; and
c) modifying the gel or the precursor thereof by reacting the functional groups of the macromer with the gel or a precursor thereof.

15. A method, comprising:
forming a hydrogel as claimed in claim 1 in the presence of cells; and
encapsulating or encasing the cells.
